(19) United States
(12) Patent Application Publication
(10) Pub. No.: US 2007/0225806 A1
(43) Pub. Date: Sep. 27, 2007

(54) Arthroplasty device

(75) Inventors: Craig M. Squires, Cordova, TN (US); Greg Marik, Germantown, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/388,938

(22) Filed: Mar. 24, 2006

(51) Int. Cl. A61F 2/44

(52) U.S. Cl. 623/17.11

(57) ABSTRACT

A prosthetic device for placement at least partially between a superior vertebra and an inferior vertebra is provided. The prosthetic device includes a single piece of material having an upper portion adapted to engage the superior vertebra, a lower portion adapted to engage the inferior vertebra, and a first motion segment having a first shape to allow movement between the superior vertebra and the inferior vertebra.

### TECHNICAL FIELD

Embodiments of the present disclosure relate generally to devices and methods for accomplishing spinal surgery, and more particularly in some embodiments, to spinal arthroplasty devices capable of being placed into the vertebral disc space.

### BACKGROUND

To date, standard treatments of the spine have not adequately addressed the need for devices, systems, and procedures to treat joint degradation. Accordingly, there is a need for improved spinal arthroplasty devices that avoid the drawbacks and disadvantages of the known implants and surgical techniques.

### SUMMARY

In one embodiment, a motion-preserving prosthetic device for use in the spine is provided.

In another embodiment, a prosthetic device for placement at least partially between a superior vertebra and an inferior vertebra is provided. The prosthetic device includes a single piece of material having an upper portion adapted to engage the superior vertebra, a lower portion adapted to engage the inferior vertebra, and a first motion segment having a first shape to allow movement between the superior vertebra and the inferior vertebra.

Additional and alternative features, advantages, uses, and embodiments are set forth in or will be apparent from the following description, drawings, and claims.

### BRIEF DESCRIPTION OF THE DRAWINGS

FIG. 1 is a perspective view of a prosthetic device according to one embodiment of the present disclosure.

FIG. 2 is a side view of the prosthetic device of FIG. 1 disposed between two adjacent vertebrae.

FIG. 3 is a perspective view of a prosthetic device according to another embodiment of the present disclosure.

FIG. 4 is a perspective view of a prosthetic device according to another embodiment of the present disclosure.

FIG. 5 is a perspective view of a prosthetic device according to another embodiment of the present disclosure.

FIG. 6 is a perspective view of a prosthetic device according to another embodiment of the present disclosure.

### DESCRIPTION

The present disclosure relates generally to vertebral reconstructive devices, and more particularly, to devices and procedures for spinal arthroplasty. For the purposes of promoting an understanding of the principles of the invention, reference will now be made to the embodiments, or examples, illustrated in the drawings and specific language will be used to describe the embodiments. It will nevertheless be understood that no limitation of the scope of the invention is intended. Any alterations and further modifications of the described embodiments, and any further applications of the principles of the invention as described herein are contemplated as would normally occur to one skilled in the art to which the invention relates.

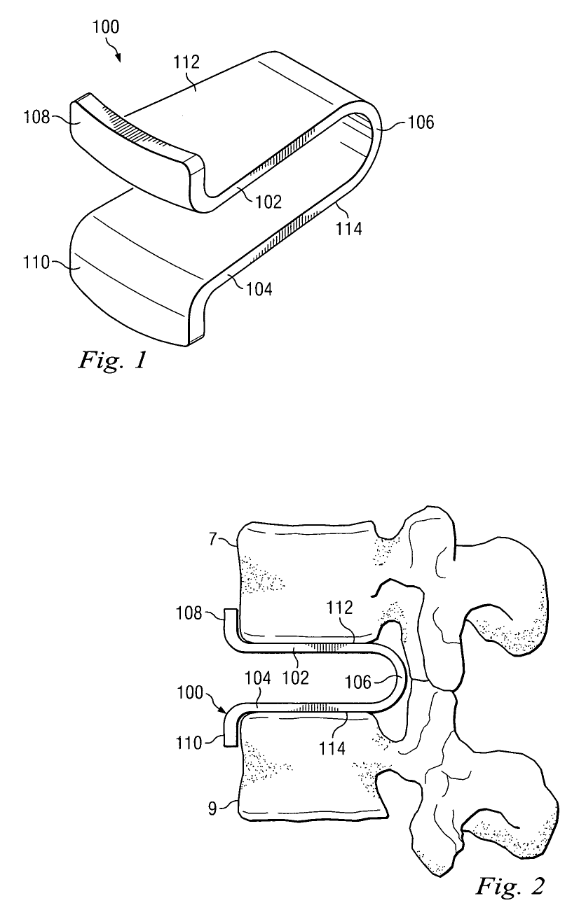

FIGS. 1 and 2 show a first exemplary embodiment of a spinal arthroplasty device according to the present disclosure. An implant 100 includes an upper portion 102, a lower portion 104, and a motion segment 106. As shown in FIG. 2, the implant 100 is adapted to fit into a disc space between a superior vertebra 7 and an inferior vertebra 9. The upper and lower portions 102, 104 include stop portions 108, 110, respectively. The stop portions 108, 110 serve to help properly position the implant 100 in the disc space by limiting how far into the disc space the implant can travel. The stop portions 108, 110 are further adapted to abut a portion of the vertebral body, such as the cortical rim, after insertion of the implant 100. Thus, the stop portions 108, 110 may be selectively curved to substantially match the curvature of the vertebral bodies of the vertebrae 7, 9.

In the present embodiment, the implant 100 is of a selected size and/or shape for the patient and the application. For example, depending upon what region of the spine—cervical, thoracic, or lumbar—the implant 100 is of a selected length L. The length L of the implant 100 may be further selected for the patient's specific size or condition. In one embodiment, the length L of the implant is such that the motion segment 106 is disposed between a midline and a posterior edge of the vertebrae 7, 9. For example, as shown in FIGS. 1-3, the motion segment 106 is desired to be positioned in a posterior portion of the implant 100. When the implant 100 is inserted between the vertebrae 7, 9, the motion segment 106 is disposed in a posterior portion of the disc space. In other embodiments, the implant 100 may include a motion segment that is adapted to be disposed between the midline and an anterior edge of the vertebrae 7, 9. The precise position desired for the motion segment 106 may be dependent upon the patient's anatomy; the geometry of the motion segment; the presence of additional motion segments; the region of the spine; the material used to form the implant 100; the presence, or lack thereof, of other artificial components in the spinal region; the surgical approach to be used; and any other factor that may influence the efficacy of implant. Similarly, a height H and a width of the implant 100 may depend on these same factors and be adjusted accordingly.

The implant 100 may be attached to the vertebrae 7, 9 utilizing a number of different attachment means including, but not limited to porous coatings, protrusions, screws, staples, tacks, adhesives, and combinations of attachment means. In some embodiments, a superior engagement surface 112 of the upper portion 102 engages the superior vertebra 7 and an inferior engagement surface 114 of the lower portion 104 engages the inferior vertebra 9. In some embodiments the engagement surfaces 112, 114 are shaped to match a contour of a surface of the vertebral endplates of the vertebrae 7, 9, respectively. Similarly, in some embodiments the stop portions 108, 110 are utilized to attach the implant 100 to the vertebrae 7, 9. The stop portions 108, 110 may include attachment means similar to engagement surfaces 112, 114. Further, in some embodiments both the engagement surfaces 112, 114 and the stop portions 108, 110 are used to attach the implant 100 to the vertebrae 7, 9.

Where the engagement surfaces 112, 114 attach the implant 100 to the vertebrae 7, 9, the engagement surfaces may include features or coatings to enhance fixation. For example, the surfaces 112, 114 may be roughened by chemical etching, bead-blasting, sanding, grinding, serrating, nanotubes, or diamond-cutting. All or a portion of the engagement surfaces 112, 114 of the upper and lower portions 102, 104 may also be coated with a biocompatible and osteoconductive material such as hydroxyapatite (HA), tricalcium phosphate (TCP), or calcium carbonate to promote bone ingrowth and fixation. Alternatively, osteoinductive coatings, such as proteins from the transforming growth factor (TGF) beta superfamily or bone-morphogenic proteins, such as BMP2 or BMP7, may be used. Other suitable features may include spikes, keels, ridges, or other surface textures designed to encourage fixation between the implant 100 and the vertebrae 7, 9.

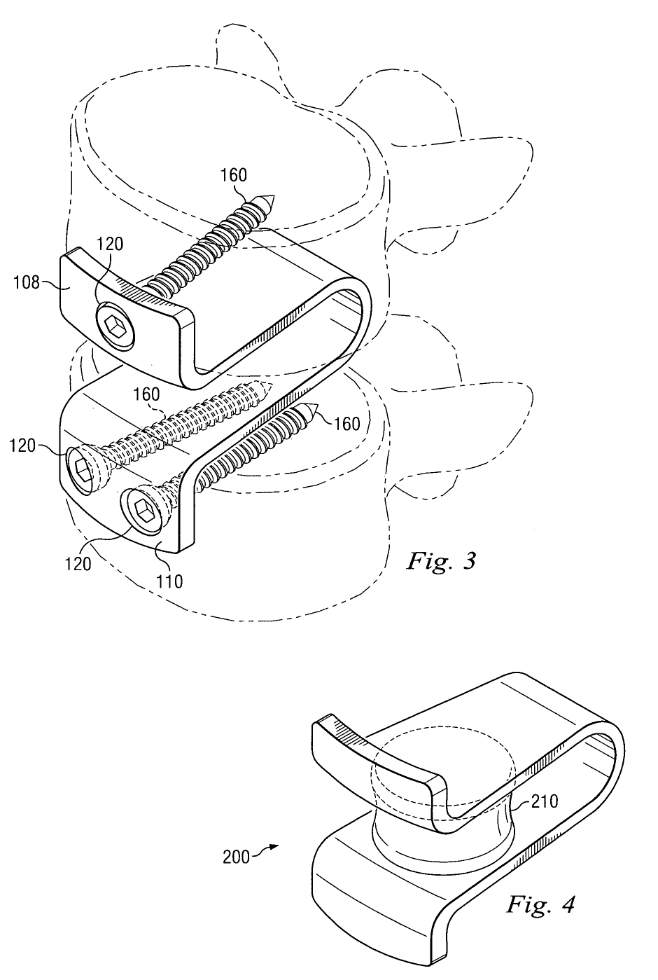

As shown in FIG. 3, in one embodiment the stop portions 108, 110 include openings or apertures 120 to facilitate attachment of the implant 100 to the vertebrae 7,9 via screws 160. The apertures 120 are oriented such that when the screws 160 are aligned with the apertures and pass through the apertures and a wall of the vertebral body to achieve strong cortical fixation. While stop portion 108 is shown as having a single aperture 120, in other embodiments the stop portion 108 has a plurality of apertures. Similarly, while stop portion 110 is shown as having two apertures 120, in other embodiments the stop portion 110 may have additional apertures or a single aperture. In some embodiments, the screws 160 are recessed with respect to an outside boundary of the apertures 120 or otherwise configured so as not to interfere with articulations, soft tissues, and neural structures. In some embodiments, the screws 160 can be constructed of a resorbable material and work in combination with another fixation method, such as one of the above-listed fixation methods associated with the engagement surfaces 112, 114. In these embodiments, the screws 160 can support the implant 100 until sufficient bone growth or other fixation has occurred on the engagement surfaces 112, 114, and afterwards be resorbed into the patient.

The implant 100 is adapted to preserve at least some motion of the vertebral joint. To this end, the implant 100 includes the motion segment 106. The motion segment 106 is of an appropriate shape or geometry to allow the implant 100 to preserve, at least partially, the motion of a joint. For example, but without limitation, the motion segment 106 may have a single curve, include multiple curves, include slits or openings, include multiple portions or parts, or consist of different types or thicknesses of materials. In this way the implant 100 may flex, compress, expand, twist, rotate, or otherwise preserve motion of the joint to a desired amount. The motion segment 106 is also shaped to provide load bearing support. In some embodiments, the load bearing support of the motion segment 106 is adapted to substantially replace the load bearing support of a natural joint. In other embodiments, the motion segment 106 may provide greater or lesser load bearing support than the natural joint.

In some embodiments the implant 100, including the upper portion 102, lower portion 104, and motion segment 106, may be formed from a single, continuous piece of material. This can be advantageous for several reasons. First, forming the implant 100 from a single piece of material significantly reduces the particle wear debris as compared to implants that have multiple components that articulate against each other, such as ball-and-socket type implants. Similarly, the implant 100 exhibits extremely low wear rates. Additionally, it can make the surgical procedure simpler as it requires implantation of only a single piece. Finally, it can make the manufacturing process relatively simple and cost effective. However, in other embodiments the implant 100 is formed from a plurality of pieces or materials. For example, the motion segment can be made of a flexible material, and the portions 102, 104 can be made of more rigid and/or porous materials.

The implant 100 may be formed of any suitable biocompatible material including metals such as cobalt-chromium alloys, titanium alloys, nickel titanium alloys, or stainless steel alloys. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); or cross-linked UHMWPE. Also, portions of the device may be formed out of ceramic. Ceramic materials such as aluminum oxide or alumina, zirconium oxide or zirconia, compact of particulate diamond, or pyrolytic carbon may also be suitable. Further, the implant 100 may be formed of multiple materials, permitting various combinations of metals, polymers, and ceramics. Finally, the implant 100 may be formed from or include a coating of a material adapted to cooperate with an imaging technique that may be used in conjunction with the implant.

The flexibility and support of the motion segment 106 may be selected based on its application. For example, where the implant 100 is adapted for use in the cervical region of the spine the motion segment 106 may allow more flexibility and provide less support than the case where the implant is adapted for use in the lumbar region of the spine. Further, the flexibility and support may be selected based on the patient's condition. For example, if the patient suffers from spondylolisthesis or scoliosis, then the motion segment 106 may be shaped to provide support and flexibility designed to facilitate correction of the condition.

The flexibility and support of the motion segment 106 may be selected by choosing the geometry of the motion segment, selecting the location of the motion segment, selecting the number of motion segments, selecting the material that the motion segment or implant 100 is made out of, or selecting the means of attachment to the vertebrae. Each of these factors may be related to each other. For example, the spring force of the material used may dictate both the shape and location of the motion segment 106. Similarly, the means of attachment may affect the shape of the motion segment or the material needed to provide the appropriate support and motion preservation. To this end, in some embodiments determining these and other attributes of the implant 100 may be facilitated by modeling the implant in a 3-D simulation of the patient's spine to determine the appropriate combinations of number of motion segments, motion segment shapes, motion segment locations, materials for the implant and motion segments, and attachment means.

Referring now to FIG. 4, shown therein is another embodiment of a spinal arthroplasty device according to the present disclosure. The implant 200 in FIG. 4 is adapted for use with a spacer 220, however implant 200 may be substantially similar to implant 100 described above. The spacer 220 may be any device or feature adapted to provide cushioning or dampening along with load bearing support. The spacer 220 can be connected to the upper and/or lower portions 102, 104. In some embodiments, the spacer 220 is a separate component that may be selectively placed between to the upper and lower portions 102, 104. Spacer 220 serves to support compressive and tensile loads on the vertebral joint while still preserving at least a limited amount of motion between vertebrae 7, 9. In some embodiments, the spacer 220 limits the amount of separation between the upper and lower portions 102, 104. The degree of allowable separation will be based upon the compression and extension characteristics of the spacer 220 and the implant 200. In some embodiments, the flexibility characteristics of the spacer 220 and the implant 200 are adapted to effectively work together to achieve the desired levels of allowed compression, extension, and axial movement. The desired levels of compression, extension, and axial movement may be tailored to each patient. For example, in some embodiments the spacer 220 may be loaded in compression or tension to counteract the patient's natural physical condition.

In some embodiments a flexible housing or sheath may be utilized to protect the spacer 220 and preserve the functioning of the system as a whole. For example, where a spring or similar device with openings is utilized as a spacer there is the possibility of interference with the function of the spring due to the body's natural processes, such as bone ingrowth, or the presence of a foreign object. The use of a flexible housing or sheath decreases the chance of such interferences.

Referring now to FIG. 5, shown therein is another embodiment of a spinal arthroplasty device according to the present disclosure. The implant 300 in FIG. 5 may be substantially similar to implants 100, 200 described above. Implant 300 includes an upper portion 302, a lower portion 304, and a motion segment 306. The upper portion 302 includes protrusions 308 adapted to engage superior vertebrae 7. Similarly, lower portion 304 includes protrusions 310 adapted to engage inferior vertebrae 9. The protrusions 308, 310 of the upper and lower portions 302, 304 may be spikes, keels, ridges, or other surface textures designed to encourage fixation between the implant 100 and the vertebrae 7, 9.

Further, the implant 300 includes a sheath 312, shown in phantom. In one embodiment, the sheath 312 is adapted to prevent foreign objects, bone engrowth, or other materials from entering the space between the upper and lower portions 302, 304. In some embodiments, the sheath 312 is filled with an injectable polymer adapted fill in the space between the upper portion 302 and the lower portion 304. In some embodiments, the injectable polymer functions as a damper between the upper and lower portions 302, 304. U.S. Patent Application Nos. 2002/0035400 to Bryan et al., 2002/0128715 to Bryan et al., and 2003/0135277 to Bryan et al. are herein incorporated by reference in their entirety. These applications provide further examples of the use of a sheath and/or injectable materials in relation to an implant.

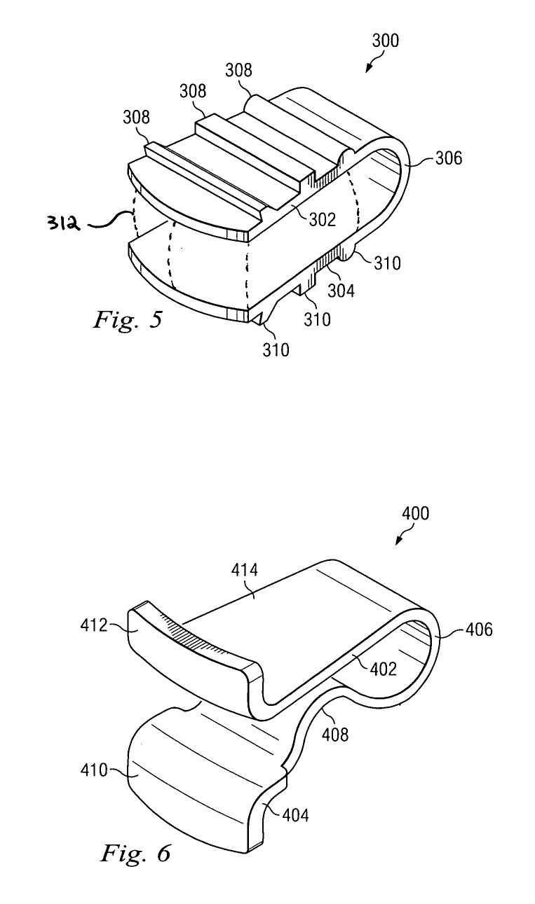

Referring now to FIG. 6, shown therein is another embodiment of a spinal arthroplasty device according to the present disclosure. The implant 400 in FIG. 6 may be substantially similar to implants 100, 200, 300 described above. Implant 400 includes an upper portion 402, a lower portion 404, and a first motion segment 406. The lower portion 404 includes a second motion segment 408 and a lower stop portion 410. The upper portion 402 includes an upper stop portion 412 and an engagement surface 414. First motion segment 406 and second motion segment 408 will be described as two separate motion segments for simplicity. However, the first and second motion segments 406, 408 may be considered parts of a single motion segment.

As in other embodiments, the motion segments 406, 408 of the implant 400 preserve motion of the vertebral joint. Further, having the first motion segment 406 disposed near a posterior portion of the implant 400 and the second motion segment 408 disposed towards the middle of the implant allows the center of rotation for the implant 400 to moved towards the middle of the implant while still providing the necessary posterior support. Also, as shown the second motion segment 408 extends upwards toward upper portion 402. Thus, in some embodiments the second motion segment 408 may serve to limit the amount of compression the implant may undergo because the travel of the upper portion 402 downward towards the lower portion 404 will be resisted as the upper portion contacts the second motion segment.

A majority of the engagement surface 414 of the upper portion 402 is adapted to engage the endplate of superior vertebrae 7. However, in some embodiments a substantial portion of the lower portion 404 does not engage the endplate of inferior vertebrae 9. This is because in these embodiments in order to facilitate motion of the joint the second motion segment 408 is not attached or connected to inferior vertebrae 9. However, in these embodiments the lower stop portion 410, other areas of the lower portion, and separate attachment means may be utilized to secure the implant 400 to the inferior vertebrae.

In some embodiments the implants 100, 200, 300, 400 described above are adapted for insertion through an anterior approach to the spine. However, in other embodiments the implants are adapted for insertion through other approaches including posterior, lateral, oblique, or any combination of these approaches. Further, in some embodiments the implants 100, 200, 300, 400 are adapted for bilateral insertion. That is, the implants will be inserted in pairs-one on each lateral side of the vertebral joint.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A prosthetic device for placement at least partially between a superior vertebra and an inferior vertebra, comprising:
a single piece of material having an upper portion adapted to engage the superior vertebra; a lower portion adapted to engage the inferior vertebra; and a first motion segment having a first shape to allow relative movement between the superior vertebra and the inferior vertebra.

2. The prosthetic device of claim 1, wherein the first motion segment is disposed at a posterior portion of the single piece of material.

3. The prosthetic device of claim 1, wherein the first shape of the first motion segment comprises a single curve.

4. The prosthetic device of claim 1, wherein the first shape of the first motion segment comprises a plurality of curves.

5. The prosthetic device of claim 1, wherein the first shape of the first motion segment includes an opening.

6. The prosthetic device of claim 1, wherein the first shape of the first motion segment is non-symmetrical.

7. The prosthetic device of claim 1, further including a second motion segment having a second shape to allow movement between the upper portion and the lower portion.

8. The prosthetic device of claim 7, wherein the first motion segment and the second motion segment function together.

9. The prosthetic device of claim 1, wherein the first motion segment is configured to create an articulation point in a posterior portion of a space between the two vertebrae.

10. The prosthetic device of claim 1, further including a stop portion adapted to engage a vertebral body of either the superior or inferior vertebra.

11. The prosthetic device of claim 10, wherein the stop portion is adapted to be secured to either the superior or inferior vertebra.

12. The prosthetic device of claim 11, the stop portion is adapted to be secured by one or more screws.

13. The prosthetic device of claim 1, wherein the upper portion includes a superior surface configured to enhance engagement with the superior vertebra.

14. The prosthetic device of claim 13, wherein the lower portion includes an inferior surface configured to enhance engagement with the inferior vertebra.

15. The prosthetic device of claim 14, wherein at least one of either the superior surface or the inferior surface includes a keel.

16. The prosthetic device of claim 14, wherein at least one of the superior surface and the inferior surface is treated with an osteoconductive material.

17. The prosthetic device of claim 14, wherein at least one of the superior surface and the inferior surface is treated with an osteoinductive material.

18. The prosthetic device of claim 1, wherein the first motion segment allows flexion motion between the superior vertebra and the inferior vertebra.

19. The prosthetic device of claim 1, wherein the first motion segment allows extension motion between the superior vertebra and the inferior vertebra.

20. The prosthetic device of claim 1, wherein the first motion segment allows axial rotation between the superior vertebra and the inferior vertebra.

21. The prosthetic device of claim 1, wherein the first motion segment allows lateral bending motion between the superior vertebra and the inferior vertebra.

22. The prosthetic device of claim 1, wherein the first motion segment is further adapted to provide load bearing support.

23. A prosthetic device for placement at least partially between a superior vertebra and an inferior vertebra, comprising:
a first portion having a first length that extends along a substantial portion of an endplate of the superior vertebra;
a second portion having a second length that extends along a substantial portion of an endplate of the inferior vertebra;
a motion portion connected between distal ends of the first and second portions, the motion portion being configured to allow relative movement between the first and second portions.

24. The prosthetic device of claim 23, wherein the first, second and motion portions are a monolithic structure.
